(19) United States
(12) Patent Application Publication
(10) Pub. No.: US 2010/0130619 A1
(43) Pub. Date: May 27, 2010

(54) PHARMACEUTICAL COMPOSITION FOR PARENTERAL ADMINISTRATION OF IDEBENONE

(76) Inventors: Joseph Schwarz, Markham (CA); Michael Weisspapir, Markham (CA)

Correspondence Address: ALPHARX INC., 168 KONRAD CRESCENT, SUITE 200, MARKHAM, L3R 9T9, CA

(21) Appl. No.: 12/276,754

(22) Filed: Nov. 24, 2008

(51) Int. Cl. A61K 31/122; C07F 9/10; A61P 25/00

(52) U.S. Cl. 514/690; 554/80

(57) ABSTRACT

The invention describes use of an injectable form of Idebenone, suitable for intravenous injection or infusion. The proposed formulation is an oil-in-water emulsion, where Idebenone is associated with the oil droplets.

### FIELD OF INVENTION

The invention relates to the field of preparation of stable formulations of Idebenone, suitable for parenteral administration. Existing oral dosage forms of Idebenone associated with high metabolization in the liver (“first pass effect”) [1] can not be administered in acute situations or in event of patient unconsciousness. Development of an injectable form of Idebenone is in high demand. [1, 2].

Antioxidants are offered to be used as protective agents to diminish brain damage caused by extended anesthesia. Various substances—antioxidants and free-radical scavengers—were tested in vitro in cell cultures, ex vivo in brain slices and in vivo in animal models. In such experiments Idebenone, 2,3-dimethoxy-5-methyl-6-(10-hydroxydecyl)-1,4-benzoquinone, demonstrated pronounced antioxidant activity and marked protection against oxidative damage to brain cells. An oral form of Idebenone is used in the treatment of cardiac muscle atrophy in Friedreich's Ataxia, as a liver protectant and to some extent, in the treatment of Alzheimer's disease [U.S. Pat. No. 5,916,925“Pharmaceutical composition for treatment of dementia” to Higuchi S., et. al.]

In a small human study of nine patients with cerebrovascular disease, 90 mg idebenone was given daily, and electroencephalograms and clinical symptoms were monitored. The results suggested that idebenone supplementation produced improvements in EEG and clinical symptoms in these patients [3].

Idebenone protects cultured cortical neurons against necrotic degeneration; it rescues cortical neurons even when applied 30 minutes after the NMDA pulse, suggesting that the drug interferes with the chain of toxic reactions triggered by an excessive stimulation of excitatory amino acid receptors [4]

Idebenone oral dosing (5 mg/kg daily for 8 weeks) in Friedreich's Ataxia patients significantly decreased a marker of oxidative DNA damage [5]. Idebenone prevented iron-induced lipo-peroxidation and cardiac muscle injury in three patients given 5 mg/kg daily for 4-9 months, resulting in a reduction of left ventricular enlargement in these patients. [6]

In experiments in cell cultures, Idebenone scavenges a variety of free radical species [7]. Idebenone can also redox couple with hypervalent species of myoglobin or hemoglobin, thus preventing lipid peroxidation promoted by these species. Likewise, Idebenone inhibits microsomal lipid peroxidation induced by ADP-iron complexes or organic hydroperoxides. In so doing, idebenone prevents the destruction of cytochrome P450, which otherwise would accompany lipid peroxidation.

It has been reported that Idebenone ameliorates learning and memory disturbances in experimental models produced by cerebral embolization, cerebral ischemia or lesions in the basal forebrain in rats, which is the area of origin of the acetylcholine neuron system projecting into the cerebral cortex, hippocampus and amygdala. In clinical tests, Idebenone was recognized to be effective in psychological deficits such as a decline in memory retention and disorientation [8].

The bioavailability of oral Idebenone is relatively high due to the polar hydrophobic nature of the molecule. However, oral administration of Idebenone accompanies a pronounced first pass metabolism of Idebenone in the liver, and only a small amount of the drug reaches the brain or other targeted organ. Additionally, the effects of oral treatment become apparent only after several weeks or even months of drug use.

An injectable form of Idebenone would overcome the first pass effect of an oral dosage form, quickly providing the required concentration in blood and brain tissues.

Nevertheless, no Idebenone dosage form suitable for parenteral delivery currently exists. The only described instance of intravenous administration of Idebenone was in an acute experiment [8] in rats, which utilized a 10% solution of polyethoxylated castor oil surfactant HCO-60. Such a delivery vehicle is not suitable for human use due to its pronounced hemolytic properties.

The low water solubility of Idebenone makes the task of producing an injectable form very difficult. Use of water miscible solvents (alcohol, propylene glycol, liquid PEG, N-methylpyrrolidone, etc.), where Idebenone dissolves well, is inappropriate due to the immediate precipitation of the drug upon contact with physiological fluids or a water phase. An inclusion complex of Idebenone with cyclodextrin has been described, but it is water dispersible, not soluble and not suitable for injection. The solubility of Idebenone in fixed oils (soy, corn, almond, etc.) is low; drug precipitates from such emulsions during storage, limiting their use for the preparation of injectable forms of rite drag.

The use of emulsions for parenteral delivery is well known. Submicron emulsions of various oils are employed in parenteral nutrition products (Intralipid™, Lipofundin™, Lipoplus®, etc.)[U.S. Pat. No. 6,008,248 “Hydrolysis-optimized lipid emulsions and use thereof, to Pscherer, G. et al.] and for the parenteral administration of poorly soluble drugs such as Diazepam (Diazemuls®) or Propofol (Diprivan®) [U.S. Pat. No. 5,908,869“Propofol compositions containing edetate” to Jones C, et al.; and U.S. Pat. No. 4,168,308 “Composition for enhancing the administration of pharmacologically active agents” to Wretlind K., et al.]

However, not one emulsion based formulation of Idebenone, suitable for parenteral administration, has ever been prepared. The only Idebenone emulsion mentioned in the literature is designed for topical application and is used in cosmetic formulations (e.g., Prevage® cream, marketed by Elizabeth Arden, which is based on U.S. Pat. No. 6,756,045“Topically applied idebenone-containing agent with protective and regenerative effect” to Neudecker B. et al.).

Unfortunately, the incorporation of Idebenone into known formulations based on published compositions and prepared according to conventional methods, provide unstable emulsions, showing phase separation and precipitation of Idebenone after several weeks in storage. Idebenone precipitates even in presence of high concentrations of surfactants.

### BRIEF DESCRIPTION OF THE INVENTION

An objective of the present invention is to provide an adequate method for protection of brain cells from functional impairment caused by extended anesthesia, using an injectable formulation of Idebenone. Surprisingly, it has been found that a stable Idebenone formulation suitable for parenteral administration, provides noticeable protection of brain tissue in the case of cellular damage. Such a formulation was prepared by using an oil-in water emulsion, made from a mixture of distinct oily components. Idebenone concentration in these formulations may vary from 0.1% to 2.5% by weight. The oil composition of the emulsion was compounded in such a manner that all incorporated Idebenone was completely dissolved in the discontinuous (oil) phase of the emulsion, avoiding drug precipitation during storage and providing a stable formulation. Various pharmaceutically acceptable oils, such as LCT oils—soya oil, canola oil, corn oil, safflower oil, almond oil, sesame oil, apricot kernel oil, fish oil; medium chain triglycerides—MET oil, other glycerides, such as acetylated monoglycerides (Myvacet, Myverol), long chain fatty acids and their esters—oleic acid, ethyl oleate, tocopherols and other oily compounds can be incorporated into oil phase of the emulsion. Natural phospholipids, such as soy or egg lecithin, as well as synthetic phospholipids, can be used as stabilizers along with a variety of physiologically acceptable surfactants of an on-ionic or anionic type, suitable for parenteral administration, e.g. polysorbates (Tween-20, Tween-80, Cremophor EL, Incrocas 35, Cremophor RH40, Poloxamer 188, TPGS, Tyloxapol, sodium oleate, sodium deoxycholate, etc.). The emulsion can be prepared by high pressure homogenization, high shear mixing or using a self-emulsifying process.

Surprisingly, it was found that Idebenone containing emulsions prepared by homogenization or by a self-emulsifying process can be further stabilized by passing through a microporous membrane with pores of 0.1 mcm or smaller. Such filtration allowed for the sterilization of the emulsion and significantly improved the stability of the formulation, preventing phase separation and idebenone precipitation.

In vivo testing of formulation were administrated via intravenous, intraperitoneal or subcutaneous injections, or added after required dilution to cell culture media during “in vitro” or “ex vivo” experiments, and demonstrated excellent biocompatibility, absence of irritation or signs of toxicity and pronounced brain tissue protection.

The following examples are intended to illustrate certain preferred embodiments of the invention and no limitation upon the invention is implied by their inclusion.

### Idebenone Oil-in-Water Emulsion Formulations

### EXAMPLE 1-12

### Idebenone in Oil-In-Water Emulsions Prepared by Homogenization

### EXAMPLE 1

### Preparation of Injectable Idebenone o/w Emulsion

Oil components of the formulation (Soybean oil NF grade, acetylated monoglycerides as Myvacet™ 9-45K and D-alpha-Tocopherol USP) were combined with lecithin. Phospholipon™ S-80 and ethoxylated castor oil (Incrocas™-35) and mixed at 40° C. for 1 hour. Idebenone was dissolved in warm mixture of oils and surfactants and then blended with water phase, comprising water, EDTA and Glycerin using high shear rotor-stator mixer (5-10,000 rpm, 2 minutes). The obtained emulsion was treated with high pressure homogenizer (e.g., Avestin™ Emulsiflex C5) at 5,000-15,000 psi (300-1000 bar) for 3-5 cycles. After cooling to room temperature, the emulsion was filtered through a sterile microporous membrane filter (0.1 mcm) in aseptic conditions and dispensed into sterile glass vials. The sealed vials were stored in refrigerator or at room temperature, protected from light.

The Idebenone content was tested using HPLC method.

Examples 2-10, were Idebenone loaded o/w emulsions prepared in similar manner, excluding example 8, where instead of high pressure homogenization the mixture of the oil aid water phases was passed through a 0.22 mcm microporous membrane 3 times. Compositions of examples 1 through 10 are presented in table 1.

TABLE 1
Idebenone in oil-in-water emulsions (Examples 1-10)

| | 1 | 2 | 3 | 4 | 5 | 6 | 7 | 8 | 9 | 10 |
|---|---|---|---|---|---|---|---|---|---|---|
| | Percentage of composition | | | | | | | | | |
| Idebenone | 1.0 | 2.0 | 1.0 | 0.25 | 0.10 | 0.35 | 2.0 | 0.5 | 0.5 | 2.0 |
| Soybean oil (LCT) | 12.5 | | 5.0 | 2.0 | | | | | | |
| Capric/caprylic | | 18.0 | 10.0 | 8.0 | 10.0 | | 12.0 | | 16.0 | 18.0 |
| triglycerides (MCT) | | | | | | | | | | |
| Tocopherol USP | 0.1 | 8.0 | | | | | 3.0 | 2.5 | | 2.0 |
| Acetylated monoglycerides | 12.5 | | 10.0 | | | 10.0 | 15.0 | | 9.0 | 10.0 |
| Ethyl oleate | | | | | | | | 5.0 | | |
| Polysorbate-80 | 2.0 | 1.0 | | | | | 0.5 | | 0.1 | 0.5 |
| TPGS | | | 0.5 | | | | | 1.0 | | |
| Tocopherol acid succinate | 0.02 | 0.01 | 0.01 | 0.01 | 0.01 | 0.02 | 0.01 | 0.01 | 0.01 | 0.01 |
| Ethoxylated castor oil | | | | | 0.25 | | | | | |
| Lecithin USP | 2.0 | 1.5 | 1.8 | 1.0 | 1.0 | 2.0 | 1.5 | 1.0 | 1.5 | 1.5 |
| (phosphatidylcholine > 70%) | | | | | | | | | | |
| Ethanol | | | 1.8 | | | | 1.5 | | | |
| Propylene glycol | | | | | | | | | 2.50 | |
| Glycerin | 2.5 | 2.25 | | 2.25 | 2.25 | | | | | 2.25 |
| Glycine | 0.5 | | | | 0.5 | | | 2.0 | | |
| EDTA disodium | 0.02 | 0.02 | 0.02 | 0.02 | 0.02 | 0.02 | 0.02 | 0.02 | 0.02 | 0.02 |
| Methyl paraben | | | | | | | | | 0.2 | |
| PURIFIED WATER | 66.86 | 67.22 | 69.87 | 86.47 | 85.87 | 87.61 | 64.47 | 87.97 | 70.57 | 63.72 |
| to 100% | | | | | | | | | | |
| Total: | 100% | 100% | 100% | 100% | 100% | 100% | 100% | 100% | 100% | 100% |

Examples 11-16 of Idebenone loaded emulsion with increased content of oil phase were prepared either by high pressure homogenization or by spontaneous emulsification of the Idebenone solution in a mixture of the oil surfactant and stabilizer after the addition of the water phase, without a homogenization step. For example 11, Idebenone was dissolved with slight heating (50-55° C.) in an oily mixture of acetylated monoglycerides (Myvacet™ 9-45K) and Vitamin E (Tocopherol mix), containing d-alpha tocopheryl polyethylene glycol 1000 succinate (Vitamin E TPGS) surfactant and soy lecithin. Propylene glycol was added to a warm solution, and then water phase, heated to 65-70° C., was added and mixed with oil composition using propeller mixer at low speed to avoid foaming. Examples 13 and 15 were prepared in the same manner as example 11, while examples 12, 14 and 16 were treated with high pressure homogenizer. The formed oil-in-water emulsions were passed through a microporous membrane filter (0.1 mcm) and stored at room temperature.

Compositions of examples 11 through 16 are presented in table 2.

TABLE 2
Idebenone in oil-in-water emulsions (Examples 11-16) with
high level of oil phase

| | 11 | 12 | 13 | 14 | 15 | 16 |
|---|---|---|---|---|---|---|
| | Percentage of composition | | | | | |
| Idebenone | 1 | 2 | 2.5 | 2.5 | 2.5 | 2.5 |
| Soybean oil (LCT) | | 28 | 2 | 2 | 2 | |
| Capric/caprylic triglycerides | | | 14 | 8 | | |
| (MCT) | | | | | | |
| Tocopherol USP | 8 | | 8 | 4 | | 2 |
| Acetylated monoglycerides | 14 | | 8 | 16 | 16 | 16 |
| Triacetin | | | | | 10 | |
| Caprylic/Capric mono/di- | | | | | | 12 |
| glycerides | | | | | | |
| Oleic acid | | 0.05 | | | | |
| Polysorbate-80 | | 5 | | 4 | 4 | |
| Solutol ® HS-15 | | | | | | 5 |
| TPGS | 5 | | | | | |
| Ethoxylated castor oil | | | 5 | | | |
| (Incrocas-35) | | | | | | |
| Lecithin USP | 1.2 | 2 | 2.5 | 2 | 2 | 2.2 |
| (phosphatidylcholine > 70%) | | | | | | |
| Ethanol | | 2 | 2.5 | | | |
| Propylene glycol | 5 | | 5 | | | 2.2 |
| Glycerin | | | | 2.25 | 2.25 | |
| Benzyl alcohol | | | | 0.5 | | |
| Dibasic sodium phosphate | | | | | 0.4 | 0.4 |
| EDTA disodium | 0.02 | 0.02 | 0.02 | 0.02 | 0.02 | 0.02 |
| Methyl paraben | 0.2 | 0.2 | 0.2 | | 0.2 | 0.2 |
| PURIFIED WATER | 65.58 | 60.73 | 40.28 | 58.73 | 60.63 | 57.48 |
| to 100% | | | | | | |
| Total: | 100% | 100% | 100% | 100% | 100% | 100% |

Formulations 1-16 are stable at room temperature for several months, with no signs of phase separation or Idebenone precipitation. The obtained oil-in-water emulsions were passed through a microporous membrane filter (0.1 mcm) without loss of Idebenone content.

1. A sterile pharmaceutical composition for parenteral administration of Idebenone, said composition comprises an oil-in-water emulsion, stabilized with pharmaceutically acceptable surfactants and stabilizers, in which Idebenone is dissolved in a water-immiscible oil phase.

2. A composition as set forth in claim 1 wherein said oil phase is comprised of pharmaceutically acceptable long chain triglycerides.

3. A composition as set forth in claim 1 wherein said oil phase is comprised of pharmaceutically acceptable medium chain triglycerides.

4. A composition as set forth in claim 1 wherein said oil phase is comprised of pharmaceutically acceptable acetylated monoglycerides or diglycerides.

5. A composition as set forth in claim 1 wherein said oil phase is comprised of mixture of long chain triglycerides and acetylated monoglycerides or diglycerides.

6. A composition as set forth in claim 1 wherein said oil phase is comprised of mixture of long chain triglycerides and medium chain triglycerides with omega-3-acid triglycerides (fish oil).

7. A composition as set forth in claim 1 wherein said oil phase may additionally be comprised of fatty acids

8. A composition as set forth in claim 1 further comprising pharmaceutically acceptable stabilizers, selected from group of natural or synthetic phospholipids.

9. A stabilizer as set forth in claim 8, wherein said phospholipid is phosphatidylcholine, phosphatidylethanolamine or a mixture thereof.

10. A stabilizer as set forth in claim 8, wherein said phospholipid is soy lecithin or egg lecithin.

11. A stabilizer as set forth in claim 8, wherein said phospholipid is hydrogenated lecithin.

12. A stabilizer as set forth in claim 8, wherein said phospholipid is synthetic phospholipid.

13. A stabilizer as set forth in claim 12, wherein said synthetic phosphatidylcholine contains PEG moiety.

14. A composition as set forth in claim 1 which may additionally contain pharmaceutically acceptable non-ionic surfactants or anionic surfactants.

15. A composition as set forth in claim 14 wherein said non-ionic surfactant is selected from a group of polyethoxylated esters or ethers of fatty acids, fatty alcohols or sorbitane or fatty acid esters of sugars and polyols.

16. A composition as set forth in claim 14 wherein said anionic surfactant is selected from a group of sodium oleate, sodium stearate or sodium deoxycholate.

17. A composition as set forth in claim 15 wherein said surfactant is polyoxyethylene sorbitan fatty acid ester.

18. A composition as set forth in claim 15 wherein said surfactant is polyethoxylated castor oil.

19. A composition as set forth in claim 15 wherein said surfactant is polyethoxylated (4-hydroxy)stearic acid Solutol® HS-15.

20. A composition as set forth in claim 15 wherein said surfactant is PEG stearate or PEG distearate.

21. A composition as set forth in claim 15 wherein said surfactant is polyoxyethylene alkylether.

22. A composition as set forth in claim 15 wherein said surfactant is d-alpha tocopheryl polyethylene glycol 1000 succinate (Vitamin E TPGS).

23. A composition as set forth in claim 15 wherein said surfactant is p-(isooctyl)polyoxyethylene phenolformaldehyde polymer (Tyloxapol®).

24. A composition as set forth in claim 15 wherein said surfactant is Poloxamer.

25. A composition as set forth in claim 1, comprised of 0.05-2.5% of idebenone, 5-60% of the oil phase, 0.1-10% of phospholipid stabilizer, 0-10% of the surfactant and 20-90% of the aqueous phase.

26. A composition as set forth in claim 25, intended for parenteral administration, non-diluted or diluted via intravenous injection or intravenous infusion.

27. A composition as set forth in claim 25, sterilized by sterile filtration.

28. A composition as set forth in claim 25, sterilized by moist heat sterilization.

29. A composition as set forth in claim 25, sterilized by gamma-irradiation.

30. A composition as set forth in claim 25, sterilized by electron beam sterilization.
